(19) United States
(12) Patent Application Publication
(10) Pub. No.: US 2009/0048514 A1
(43) Pub. Date: Feb. 19, 2009

(54) DEVICE FOR ULTRASOUND MONITORED TISSUE TREATMENT

(75) Inventors: Haim Azhari, Doar-Na Misgav (IL); Jacob Benarie, Haifa (IL); Yossi Gross, Moshav Mazor (IL); Liat Tsoref, Tel Aviv (IL)

Correspondence Address: DARBY & DARBY P.C., P.O. BOX 770, Church Street Station, New York, NY, 10008-0770, US

(73) Assignee: Slender Medical Ltd., Herzliya (IL)

(21) Appl. No.: 12/207,043

(22) Filed: Sep. 9, 2008

Related U.S. Application Data
(63) Continuation of application No. PCT/IL2007/000307, filed on Mar. 8, 2007.
Application No. PCT/IL2007/000307 is a continuation-in-part of application No. 11/651,198, filed on Jan. 8, 2007.
Application No. 11/651,198 is a continuation-in-part of application No. 11/653,115, filed on Jan. 12, 2007.
(60) Provisional application No. 60/780,772, filed on Mar. 9, 2006.
(60) Provisional application No. 60/809,577, filed on May 30, 2006.
(60) Provisional application No. 60/860,635, filed on Nov. 22, 2006.

(51) Int. Cl. A61B 8/00; A61N 7/00

(52) U.S. Cl. 600/439; 601/2

(57) ABSTRACT

Apparatus is provided for lipolysis and body contouring of a subject. The apparatus includes a housing adapted for placement on tissue of the subject. The apparatus also includes a plurality of acoustic elements disposed at respective locations with respect to the housing, including at least a first and a second subset of the acoustic elements, wherein the first subset is configured to transmit energy in a plane defined by the housing, such that at least a portion of the transmitted energy reaches the second subset. Other embodiments are also described.

### CROSS-REFERENCES TO RELATED APPLICATIONS

The present application:

(a) claims the priority of:
U.S. Provisional Patent Application 60/780,772 to Azhari et al., filed Mar. 9, 2006, entitled, “A method and system for lypolysis and body contouring,”
U.S. Provisional Patent Application 60/809,577 to Azhari et al., filed May 30, 2006, entitled, “A device for ultrasound monitored tissue treatment,” and
U.S. Provisional Patent Application 60/860,635 to Azhari et al., filed Nov. 22, 2006, entitled, “Cosmetic tissue treatment using ultrasound,” and

(b) is a continuation-in-part of and claims the priority of:
U.S. patent application Ser. No. 11/651,198 to Azhari et al. filed Jan. 8, 2007, entitled, “A device for ultrasound monitored tissue treatment,” and
U.S. patent application Ser. No. 11/653,115 to Azhari et al., filed Jan. 12, 2007, entitled, “A method and system for lipolysis and body contouring.”

Each of the above applications is incorporated herein by reference.

### FIELD OF THE INVENTION

The present invention relates in general to tissue treatment by application of energy thereto, and specifically to the monitoring and applying of ultrasound to skin.

### BACKGROUND OF THE INVENTION

Systems for applying energy to biological tissue are well known. Such energy application may be intended to heal injured tissue, ablate tissue, or improve the appearance of tissue. Energy may be applied in different forms, such as radiofrequency, laser, or ultrasound.

US Patent Application Publication 2004/0039312 to Hillstead et al., which is incorporated herein by reference, describes a system for the destruction of adipose tissue utilizing high intensity focused ultrasound (HIFU) within a patient's body. The system is described as comprising a controller for data storage and the operation and control of a plurality of elements. One element is described as a means for mapping a human body to establish three dimensional coordinate position data for existing adipose tissue. The controller is able to identify the plurality of adipose tissue locations on said human body and establish a protocol for the destruction of the adipose tissue. A HIFU transducer assembly having one or more piezoelectric element(s) is used along with at least one sensor, wherein the sensor provides feedback information to the controller for the safe operation of the piezoelectric element(s). The sensor is electronically coupled to the controller, and the controller provides essential treatment command information to one or more piezoelectric element(s) based on positioning information obtained from the three dimensional coordinate position data.

U.S. Pat. No. 6,500,141 to Irion et al., which is incorporated herein by reference, describes an apparatus for treating body tissue, in particular superficial soft tissue, with ultrasound, comprising an ultrasonic generation unit and an applicator, by means of which the ultrasound can be irradiated from an applicator surface facing the body surface from outside through the body surface into the body tissue. A suction apparatus for sucking in the body surface against the applicator surface is provided. An apparatus for treating body tissue including superficial soft tissue, with ultrasound, is described as comprising an ultrasonic generation unit and an applicator having an applicator surface facing the body surface from which the ultrasound can be irradiated through the body surface into the body tissue. A suction apparatus is provided for taking in the body surface against the applicator surface which is curved inwardly.

U.S. Pat. No. 5,601,526 to Chapelon et al., which is incorporated herein by reference, describes a method and apparatus for performing therapy using ultrasound. The apparatus is described as using a treatment device having at least one piezoelectric transducer element to supply ultrasonic waves focused onto a focal point or region that determines the tissue zone submitted to therapy. The treatment device, which is controlled by a control device, supplies two types of ultrasonic waves, the first one being thermal waves that produce a predominantly thermal effect on the tissue being treated and the second one being cavitation waves that produce a predominantly cavitation effect on the tissue to be treated. A therapy method is described, using ultrasound for the purpose of destroying a target. The target includes tissue, which may be located inside a body of a mammal. Ultrasonic waves are focused onto a focal point or region. A tissue zone to be submitted to the therapy is determined. Ultrasonic waves are supplied to the target. The ultrasonic waves of two types: thermal waves, for producing a predominantly thermal effect on tissue to be treated, and cavitation waves, for producing a predominantly cavitation effect on the tissue to be treated. The two types of waves are applied for a time sufficient to effect therapy by destroying at least a portion of the tissue, and the thermal ultrasonic waves are supplied at least at a beginning of treatment. In an embodiment, the ultrasonic waves are supplied after an adjustable predetermined time interval for allowing preheating of the tissue to be treated.

PCT Publication WO 06/018837 to Azhari et al., which is incorporated herein by reference, describes a method of damaging a target tissue of a subject. The method is described as comprising: (a) imaging a region containing the target tissue; (b) determining a focal region of a damaging radiation; (c) positioning the focal region onto the target tissue; and (d) damaging the target tissue by an effective amount of the damaging radiation. The determination of the focal region is described by delivering to the region bursts of ultrasonic radiation from a plurality of directions and at a plurality of different frequencies, and passively scanning the region so as to receive from the region ultrasonic radiation having at least one frequency other than the plurality of different frequencies.

PCT Publication WO 06/080012 to Kreindel, which is incorporated herein by reference, describes a system and method for heating a tissue volume under a skin surface of an individual from an initial temperature to a predetermined treatment temperature in the range of 42 C-60 C. The method is described as comprising applying electrodes to the skin surface and providing from the electrodes a continuous wave radiofrequency (RF) energy or a quasi-continuous wave RF energy, where the RF energy has a power selected to heat the tissue volume to the final temperature in an amount of time exceeding 0.5 sec. The system is described as comprising electrodes and an RF generator configured to provide a continuous wave RF voltage energy or a quasi-continuous wave RF voltage across the electrodes where the RF energy has a power selected to heat the tissue volume to the final temperature in an amount of time exceeding 0.5 sec.

US Patent Application Publications 2005/0154308, 2005/0154309, 2005/0193451, 2004/0217675, 2005/0154295, 2005/0154313, 2005/0154314, 2005/0154431, 2005/0187463, 2005/0187495, 2006/0122509, 2003/0083536, 2005/0261584, 2004/0215110, 2006/0036300, 2002/0193831, and 2006/0094988, U.S. Pat. Nos. 5,143,063, 6,730,034, 6,450,979, 6,113,558, 6,607,498, 6,626,854, 6,645,162, and 6,971,994, and PCT Patent Publications WO/2000/053263, and WO/2005/074365 are incorporated herein by reference.

The following articles, which are incorporated herein by reference, may be of interest:

Moran CM et al., “Ultrasonic propagation properties of excised human skin,” Ultrasound Med Biol. 21(9):1177-90 (1995)

Akashi N et al., “Acoustic properties of selected bovine tissue in the frequency range 20-200 MHz,” J Acoust Soc Am. 98(6):3035-9 (1995)

### SUMMARY OF THE INVENTION

In some embodiments of the invention, cosmetic and/or medical apparatus is provided which comprises a tissue monitoring system and a tissue treatment system. The monitoring system assesses a state of tissue of a subject, and the treatment system applies a treatment to the tissue. The treatment typically includes various cosmetic treatments (e.g., body contouring by lipolysis, hair removal, wrinkle and face lift, or face-localized molding of adipose tissue). Typically, the monitoring and treatment occur in alternation, until the monitoring system determines that the treatment has been completed. For some applications, one of the systems comprises a housing, and the tissue of the subject is sucked at least partially into the housing, to allow the system to monitor or treat (as appropriate) the tissue that has been sucked into the housing. In this case, the system typically transmits ultrasound energy that is designated to remain in large part within the housing and tissue therein, and generally not to affect tissue outside of the housing.

As appropriate for a given application, the system comprising the housing may be the monitoring system, the treatment system, or both the monitoring system and the treatment system.

In an embodiment, the housing comprises a plurality of acoustic elements, e.g., ultrasound transducers, arranged in a circle (or other typically but not necessarily closed shape). The transducers are positioned such that ultrasound energy transmitted by the transducers remains generally within a plane defined by the circle. Similarly, in embodiments in which the monitoring system comprises the housing, the transducers are typically disposed such that they are optimized to receive ultrasound energy coming generally from within the plane.

In an embodiment, a first subset of the plurality of acoustic elements is disposed at one location of the housing and a second subset of acoustic elements is disposed at a second location of the housing. Each subset contains one or more acoustic elements designated to transmit energy and/or one or more acoustic elements designated to receive and/or reflect energy. The acoustic elements configured to receive energy comprise transducers which convert the energy into information capable of being processed by a processor typically located remotely from the acoustic elements, enabling reflected, scattered, or through-transmitted energy to be analyzed.

Treatments using the treatment system may include, as appropriate, causing heating, tissue damage, thermal ablation, acoustic streaming, mechanical irritation, cell structure alteration, augmented diffusion, and/or a cavitation effect.

Typically, the treatment system comprises circuitry for configuring the applied energy as high intensity focused ultrasound (HIFU), using techniques known in the art.

In some embodiments, the housing comprises two generally-parallel cylinders spaced at a predetermined distance from one another so as to define a plane between the cylinders, and a support element connected to both cylinders. For some applications, an electromechanical system is configured to vary the distance between the cylinders and/or rotate the cylinders after the housing comes in contact with skin of the subject. Consequently, the tissue is pinched and drawn at least partially into the plane to be subsequently monitored or treated (as appropriate) by the acoustic elements.

For some applications, the housing is flexible, e.g., to allow the treatment of limbs or other curved body parts. Alternatively, the housing is generally rigid.

For some applications, the housing comprises a flexible cuff configured to surround a limb of the subject designated for treatment. The subsets of acoustic elements are typically arranged around the cuff on a circle defined by the cuff. For some applications, the acoustic elements are configured to remain fixed at their respective locations with respect to the cuff, while the cuff moves about the limb. For other applications, an electromechanical system moves at least a portion of the acoustic elements to different locations on the cuff.

In an embodiment, the monitoring system generally continuously generates acoustic maps or images, depicting changes occurring during a treatment of the tissue within the housing. For some applications, this allows an operator of the treatment system to monitor the progress of a treatment, and to alter a parameter of the treatment in response thereto. Such a parameter may include, for example, a location of a focus of the HIFU, a positioning of the housing on the subject's skin, or a strength of the applied energy. Alternatively or additionally, the treatment system and monitoring system operate in a closed loop fashion, whereby an output of the monitoring system (e.g., a location of fatty tissue) is used as an input parameter to the treatment system, such that the treatment system can adjust its operating parameters in response to the output of the monitoring system (and, for example, heat the fatty tissue).

In an embodiment, the apparatus comprises a tracking system comprising reference sensors configured to track progress of treatments conducted on different days, or during the same procedure, by registering and recording the spatial location of the treated tissue. Typically, the spatial localization is achieved in comparison to corresponding predefined anatomical locations of the subject with respect to the housing. Alternatively, the spatial localization corresponds to coordinates in a room with respect to the housing.

There is therefore provided, in accordance with an embodiment of the invention, apparatus, including:

a housing, adapted for placement on tissue of a subject; and

a plurality of transducers, disposed at respective locations with respect to the housing, and configured to transmit energy towards each other, in a plane defined by the housing.

In an embodiment, the plurality of transducers are disposed with respect to the housing so as to define a ring of transducers.

In an embodiment, the apparatus includes a source of suction configured to draw the tissue into the housing, and the transducers are disposed with respect to the housing so as to direct the energy into the tissue within the housing.

In an embodiment, the apparatus includes a pinching tool, configured to draw the tissue into the housing by pinching the tissue, and the transducers are disposed with respect to the housing so as to direct the energy into the tissue within the housing.

In an embodiment, the transducers are configured to substantially avoid transmitting energy out of the plane.

In an embodiment, the transducers include ultrasound transducers.

In an embodiment, the tissue includes skin of the subject, and wherein the transducers are configured to transmit the energy through the skin.

In an embodiment, the housing is flexible at least in part, and configured to flex to match a shape of the tissue.

In an embodiment, the housing is generally rigid.

In an embodiment, the apparatus is configured to apply a treatment to the tissue without monitoring a state of the tissue.

In an embodiment, the apparatus is configured to apply a treatment to the tissue by elevating a temperature of the tissue by less than 10 C.

In an embodiment, the apparatus is configured to elevate the temperature by less than 5 C.

In an embodiment, the apparatus is configured to receive energy in response to the transmitted energy, and to monitor a state of the tissue in response to the received energy.

In an embodiment, the plurality of transducers are configured to cycle repeatedly between (a) applying a treatment to the tissue in response to the monitored state of the tissue, and (b) monitoring the state of the tissue following (a).

In an embodiment, the apparatus includes a robotic system configured to move the housing in response to a parameter of the monitored state of the tissue.

In an embodiment, the plurality of transducers are not configured to apply a treatment to the tissue in response to the monitored state of the tissue.

In an embodiment, the plurality of transducers are configured to apply a treatment to the tissue in response to the monitored state of the tissue.

In an embodiment, the transmitted energy has a first energy level associated therewith, and, in applying the treatment, the transducers are configured to transmit energy at a second energy level that is higher than the first energy level.

In an embodiment, in monitoring the state of the tissue, the apparatus identifies a concentration of fat in the tissue.

In an embodiment, the apparatus is configured to drive the plurality of transducers to direct energy towards the concentration of fat.

In an embodiment, the plurality of transducers are configured to transmit treatment energy configured for lipolysis of adipose tissue.

In an embodiment, the plurality of transducers are configured to configure the treatment energy for removal of hair of the subject.

In an embodiment, the plurality of transducers are configured to configure the treatment energy for treatment of wrinkles of the subject.

In an embodiment, the plurality of transducers are configured to configure the treatment energy for at least one cosmetic treatment selected from the group consisting of: neck lift, mentoplasty, body contouring, and molding of adipose tissue.

In an embodiment, plurality of transducers are configured to configure the treatment energy for a face-localized molding of adipose tissue.

There is further provided, in accordance with an embodiment of the present invention, apparatus including:

a housing adapted for placement on tissue of a subject; and

a plurality of acoustic elements disposed at respective locations with respect to the housing, including at least a first and a second subset of the acoustic elements, the first subset is configured to transmit energy in a plane defined by the housing, such that at least a portion of the transmitted energy reaches the second subset.

In an embodiment, the first subset is configured to elevate a temperature of the tissue to at least 57 C.

In an embodiment, the first subset is configured to elevate a temperature of the tissue by between 10 C and 20 C.

In an embodiment, at least one of the acoustic elements of the second subset includes an ultrasound reflector.

In an embodiment, the tissue includes skin of the subject, and the first subset is configured to transmit the energy through the skin.

In an embodiment, the housing is flexible at least in part, and configured to flex to match the shape of the tissue.

In an embodiment, the housing is generally rigid.

In an embodiment, the apparatus includes a source of suction configured to draw the tissue into the housing, and the plurality of acoustic elements are disposed with respect to the housing so as to direct the energy to the tissue within the housing.

In an embodiment, the housing is configured to pinch a portion of the tissue to draw the portion into the plane.

In an embodiment, the first subset is configured to substantially avoid transmitting energy out of the plane.

In an embodiment, the first subset includes ultrasound transducers.

In an embodiment, at least one of the acoustic elements of the second subset includes an ultrasound transducer.

In an embodiment, the ultrasound transducer is configured to transmit energy in the plane, such that at least a portion of the energy transmitted by the ultrasound transducer reaches the first subset.

In an embodiment, the first subset is configured to transmit treatment energy.

In an embodiment, the first subset is configured to configure the treatment energy for lipolysis of adipose tissue.

In an embodiment, the first subset is configured to generate an imploding wave in the plane by transmitting high intensity waves.

In an embodiment, the first subset is configured to generate an imploding cylindrical wave in the plane by transmitting high intensity waves.

In an embodiment, the first subset is configured to configure the treatment energy for removal of hair of the subject.

In an embodiment, the first subset is configured to configure the treatment energy for treatment of wrinkles of the subject.

In an embodiment, the first subset is configured to configure the treatment energy for at least one cosmetic treatment selected from the group consisting of: neck lift, mentoplasty, body contouring, and molding of adipose tissue.

In an embodiment, the first subset is configured to configure the treatment energy for a face-localized molding of adipose tissue.

In an embodiment, the first subset is configured to elevate a temperature of the tissue by less than 10 C.

In an embodiment, the first subset is configured to elevate the temperature by less than 5 C.

In an embodiment, the apparatus includes an electromechanical system configured to move at least some of the plurality of acoustic elements with respect to the plane.

In an embodiment, in moving the elements, the electromechanical system is configured to move at least a portion of the acoustic elements of one of the subsets at the same time.

In an embodiment, the housing includes the electromechanical system.

In an embodiment, the electromechanical system is configured to move the housing.

In an embodiment, the electromechanical system is configured to maintain a predetermined distance between the first subset and the second subset.

In an embodiment, the electromechanical system is configured to vary a distance between the first subset and the second subset.

In an embodiment, the electromechanical system is configured to draw the tissue into the housing by pinching the tissue, and the plurality of acoustic elements are disposed with respect to the housing so as to direct the energy to the tissue within the housing.

In an embodiment, the plurality of acoustic elements are configured to monitor a parameter of the tissue.

In an embodiment, the apparatus includes a processing unit configured to generate a computed tomography image of the tissue in response to the energy received by the second subset.

In an embodiment, the parameter of the tissue includes fat content.

In an embodiment, a first portion of the plurality of acoustic elements is configured to transmit treatment energy, and at least some of the plurality of acoustic elements are configured to monitor an alteration of the parameter in response to the treatment energy.

In an embodiment, the apparatus includes an energy source that is not an acoustic element from the first or second subsets of acoustic elements, and the energy source is configured to transmit treatment energy in response to the monitoring.

In an embodiment, the energy source is configured to configure the treatment energy for lipolysis of adipose tissue.

In an embodiment, the energy source is configured to generate an imploding wave in the plane by transmitting high intensity waves.

In an embodiment, the energy source is configured to generate an imploding cylindrical wave in the plane by transmitting high intensity waves.

In an embodiment, the energy source is configured to configure the treatment energy for removal of hair of the subject.

In an embodiment, the energy source is configured to configure the treatment energy for treatment of wrinkles of the subject.

In an embodiment, the energy source is configured to configure the treatment energy for at least one cosmetic treatment selected from the group consisting of: neck lift, mentoplasty, body contouring, and molding of adipose tissue.

In an embodiment, the energy source is configured to configure the treatment energy for a face-localized molding of adipose tissue.

In an embodiment, the energy source is configured to transmit treatment energy in conjunction with the monitoring.

In an embodiment, the energy source is configured to apply the treatment to the tissue by elevating a temperature of the tissue by less than 10 C.

In an embodiment, the energy source is configured to elevate the temperature by less than 5 C.

In an embodiment, the energy source is configured to configure the treatment energy for a face-localized molding of adipose tissue.

In an embodiment, the housing includes a cuff, and the plurality of acoustic elements are coupled to the cuff.

In an embodiment, the plurality of acoustic elements are disposed with respect to the cuff so as to define a ring of acoustic elements.

In an embodiment, the tissue includes tissue of a limb of the subject, and the cuff is configured to surround the limb.

In an embodiment, the apparatus includes a motor coupled to the cuff, configured to move at least some of the plurality of acoustic elements.

In an embodiment, the plurality of acoustic elements are disposed at fixed locations with respect to the cuff.

In an embodiment, the plurality of acoustic elements is configured to effect contouring of the body of the subject.

In an embodiment, the apparatus includes a sensor coupled to the housing, configured to identify a location of the housing.

In an embodiment, the apparatus includes a sensor coupled to the housing, configured to identify a location of tissue, and a processing unit, configured to compare the location with a previously-stored location of tissue.

There is additionally provided, in accordance with an embodiment of the present invention, apparatus, including:

a cuff housing configured to surround a portion of a limb of a subject; and

a plurality of acoustic elements disposed at respective locations with respect to the cuff housing, including at least a first and a second subset of the acoustic elements, the first subset is configured to transmit energy such that at least a portion of the transmitted energy reaches the second subset.

In an embodiment, the plurality of acoustic elements are disposed with respect to the cuff housing so as to define a ring of acoustic elements.

In an embodiment, the plurality of acoustic elements are disposed at fixed locations with respect to the cuff housing.

In an embodiment, at least one of the acoustic elements of the second subset includes an ultrasound reflector.

In an embodiment, the cuff housing is flexible at least in part, and configured to flex to match the shape of the portion of the limb.

In an embodiment, the cuff housing is generally rigid.

In an embodiment, the first subset includes ultrasound transducers.

In an embodiment, at least one of the acoustic elements of the second subset includes an ultrasound transducer.

In an embodiment, the portion of the limb includes skin of the subject, and the ultrasound transducer is configured to transmit energy to the skin, such that at least a portion of the energy transmitted by the ultrasound transducer reaches the first subset.

In an embodiment, the first subset is configured to transmit treatment energy to the portion of the limb.

In an embodiment, the first subset is configured to configure the treatment energy for lipolysis of adipose tissue.

In an embodiment, the first subset is configured to configure the treatment energy for removal of hair of the subject.

In an embodiment, the first subset is configured to configure the treatment energy for treatment of wrinkles of the subject.

In an embodiment, the first subset is configured to configure the treatment energy for at least one cosmetic treatment selected from the group consisting of: neck lift, mentoplasty, body contouring, and molding of adipose tissue.

In an embodiment, the first subset is configured to apply the treatment energy to the portion of the limb by elevating a temperature of fat in the portion of the limb by less than 10 C.

In an embodiment, the first subset is configured to elevate the temperature by less than 5 C.

In an embodiment, the apparatus includes an electromechanical system configured to move at least some of the plurality of acoustic elements with respect to the portion of the limb.

In an embodiment, in moving the elements, the electromechanical system is configured to move at least a portion of the acoustic elements of one of the subsets at the same time.

In an embodiment, the cuff housing includes the electromechanical system.

In an embodiment, the electromechanical system is configured to move the cuff housing.

In an embodiment, the electromechanical system is configured to maintain a predetermined distance between the first subset and the second subset.

In an embodiment, the electromechanical system is configured to vary a distance between the first subset and the second subset.

In an embodiment, the plurality of acoustic elements are configured to monitor a parameter of the portion of the limb of the subject.

In an embodiment, the parameter of the portion of the limb includes fat content, and the plurality of acoustic elements are configured to monitor the fat content.

In an embodiment, a first portion of the plurality of acoustic elements is configured to transmit treatment energy, and a second portion of the plurality of acoustic elements is configured to monitor an alteration of the parameter in response to the treatment energy.

In an embodiment, the apparatus includes an energy source that is not an acoustic element from the first or second subsets of acoustic elements, and the energy source is configured to transmit treatment energy in response to the monitoring.

In an embodiment, the energy source is configured to configure the treatment energy for lipolysis of adipose tissue.

In an embodiment, the energy source is configured to configure the treatment energy for removal of hair of the subject.

In an embodiment, the energy source is configured to configure the treatment energy for treatment of wrinkles of the subject.

In an embodiment, the energy source is configured to configure the treatment energy for at least one cosmetic treatment selected from the group consisting of: neck lift, mentoplasty, body contouring, and molding of adipose tissue.

In an embodiment, the energy source is configured to transmit treatment energy in conjunction with the monitoring.

In an embodiment, the plurality of acoustic elements is configured to effect contouring of the body of the subject.

In an embodiment, the apparatus includes at least one sensor configured to track the contouring.

In an embodiment, the at least one sensor is coupled to the cuff housing.

In an embodiment, the at least one sensor is configured to localize the portion of the limb of the subject with respect to predefined locations of a body of the subject.

In an embodiment, the apparatus includes a plurality of reference elements that are configured to be disposed at respective coordinates in a room, and to be in communication with the sensor.

In an embodiment, the plurality of reference elements are configured to localize the portion of the limb of the subject with respect to the coordinates of the room.

There is still additionally provided, in accordance with an embodiment of the present invention, apparatus, including:

a housing adapted for placement on tissue of a subject; and

a plurality of energy transducers disposed at respective locations with respect to the housing, including at least a first and a second subset of the energy transducers, the first subset is configured to transmit energy in a plane defined by the housing, such that at least a portion of the transmitted energy reaches the second subset.

In an embodiment, the first subset is configured to substantially avoid transmitting energy out of the plane.

In an embodiment, the first subset is configured to emit laser energy into the plane.

In an embodiment, the first subset is configured to transmit visual energy into the plane.

In an embodiment, the first subset is configured to transmit radiofrequency energy into the plane.

In an embodiment, the first subset is configured to transmit electromagnetic radiation into the plane.

In an embodiment, the first subset is configured to transmit microwave radiation into the plane.

In an embodiment, the apparatus includes a source of suction configured to draw the tissue into the housing, and the plurality of energy transducers are disposed with respect to the housing so as to direct the energy to the tissue within the housing.

In an embodiment, the housing includes a cuff configured to surround a limb of the subject, and the plurality of energy transducers are coupled to the cuff.

In an embodiment, the first subset is configured to transmit treatment energy configured for lipolysis of adipose tissue.

In an embodiment, the first subset is configured to configure the treatment energy for removal of hair of the subject.

In an embodiment, the first subset is configured to configure the treatment energy for treatment of wrinkles of the subject.

In an embodiment, the first subset is configured to configure the treatment energy for at least one cosmetic treatment selected from the group consisting of: neck lift, mentoplasty, body contouring, and molding of adipose tissue.

In an embodiment, the first subset is configured to configure the treatment energy for a face-localized molding of adipose tissue.

In an embodiment, the first subset is configured to transmit ultrasound energy into the plane.

In an embodiment, the apparatus is configured to treat the tissue of the subject, and the apparatus is configured to monitor the treatment by transmitting ultrasound energy.

There is also provided, in accordance with an embodiment of the invention, apparatus, including:

a housing, adapted for placement on tissue of a subject; and

a plurality of transducers, disposed at respective locations with respect to the housing, and configured to transmit energy towards each other, in a vicinity of a plane defined by the housing.

In an embodiment, the plurality of transducers are configured to transmit energy to a vicinity below the plane defined by the housing.

In an embodiment, the plurality of transducers are configured to transmit energy to at most 10 degrees below the plane defined by the housing.

In an embodiment, the plurality of transducers are configured to transmit energy to at most 5 degrees below the plane defined by the housing.

In an embodiment, the apparatus is configured to receive energy in response to the transmitted energy, and to monitor a state of the tissue in response to the received energy.

In an embodiment, the plurality of transducers are configured to cycle repeatedly between (a) applying a treatment to the tissue in response to the monitored state of the tissue, and (b) monitoring the state of the tissue following (a).

The present invention will be more fully understood from the following detailed description of embodiments thereof, taken together with the drawings, in which:

### BRIEF DESCRIPTION OF THE DRAWINGS

FIG. 1 is a schematic illustration of an ultrasound device, in accordance with an embodiment of the present invention;

FIG. 2 is a schematic illustration of apparatus comprising the ultrasound device of FIG. 1, positioned on tissue of a subject, in accordance with an embodiment of the present invention;

FIG. 3 is a cross-sectional view of a portion of the apparatus shown in FIG. 2, in accordance with an embodiment of the present invention;

FIG. 4 is a schematic illustration of operation of the apparatus of FIG. 2 in a monitoring mode, in accordance with an embodiment of the present invention; and

FIG. 5 is a schematic illustration of operation of the apparatus of FIG. 2 in a treatment mode, in accordance with an embodiment of the present invention.

FIG. 6 is a schematic illustration of a monitoring device positioned on tissue of a subject, and a treatment device, in accordance with an embodiment of the present invention;

FIGS. 7A and 7B are schematic illustrations of the monitoring device of FIG. 6 comprising the treatment device, in accordance with respective embodiments of the present invention;

FIG. 8 is a schematic illustration of a monitoring device positioned on tissue of a subject and a treatment device, in accordance with another embodiment of the present invention;

FIGS. 9A and 9B are schematic illustrations of the monitoring device of FIG. 8 comprising the treatment device, in accordance with respective embodiments of the present invention;

FIGS. 10 and 11 are schematic illustrations of a tracking system associated with the devices of FIGS. 1-9B, in accordance with an embodiment of the present invention; and

FIGS. 12 and 13 are graphs of transmitted pressure amplitude and time of treatment, in accordance with an embodiment of the present invention.

### DETAILED DESCRIPTION OF EMBODIMENTS OF THE INVENTION

FIG. 1 is a schematic illustration of an ultrasound device 8, in accordance with an embodiment of the present invention. Ultrasound device 8 comprises a plurality of ultrasound transducers 12, coupled to a support structure. The support structure maintains the transducers in a desired relationship with respect to each other, such as in a ring 10 of ultrasound transducers 12 (as shown), in another closed configuration (e.g., an ellipse), or in an open configuration (e.g., a C-shaped configuration, not shown). For some applications, the support structure comprises a rigid material, to rigidly maintain the desired relationship of the ultrasound transducers with respect to each other. For other applications, the support structure is at least somewhat flexible, to enable the ultrasound transducers to maintain suitable acoustic coupling with rounded tissue of a subject (such as a limb).

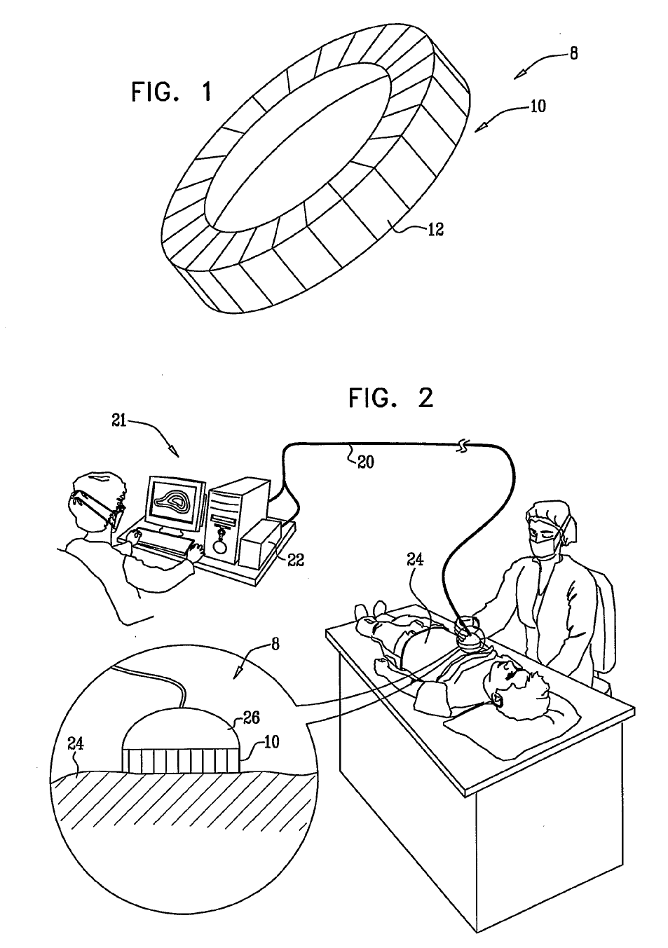

FIG. 2 is a schematic illustration of ultrasound device 8, coupled to a cover 26, and positioned on tissue 24 of a subject, in accordance with an embodiment of the present invention. Ultrasound transducers 12 of ring 10 are typically connected via coupling lines 20 to a workstation 21 which is configured to drive and receive data from ultrasound transducers 12. Workstation 21 processes signals from transducers 12 in order to generate acoustic maps or images of tissue 24 that is enclosed within ring 10. The resultant maps or images indicate whether a desired extent of treatment has been obtained (e.g., a level of damage to tissue), and guide further treatment.

It is noted that although some embodiments of the present invention are described herein with respect to generally closed-loop operation of ultrasound device 8, the scope of the present invention includes the use of ultrasound device 8 only for monitoring the tissue, while, for example, another device (e.g., a prior art ultrasound device) applies a treatment. Similarly, the scope of the present invention includes the use of ultrasound device 8 only for treating the tissue, while, for example, another device (e.g., a prior art ultrasound device) monitors the progress of the treatment. Alternatively, only monitoring is performed, or only treatment is performed.

An electromechanical system 22 is typically connected to cover 26 via coupling lines 20, to generate suction under cover 26. Optionally, electromechanical system 22 dispenses ultrasound gel to enhance acoustic coupling with the tissue. Alternatively or additionally, electromechanical system 22 dispenses water for cooling the device or tissue. Further alternatively or additionally, cover 26, an inner portion of ring 10, or another component comprises a reservoir (not shown) of water and/or gel, for dispensing by an operator during a procedure.

Reference is now made to FIGS. 2 and 3. FIG. 3 is a schematic illustration of a portion of ultrasound device 8, in accordance with an embodiment of the present invention. In an embodiment, the operator (as shown) or a robotic system moves ultrasound device 8 to different sites on tissue 24. For example, the tissue may be skin overlying a significant deposit of fat, and the patient may be undergoing a cosmetic procedure to remove the fat. Vacuum is applied by electromechanical system 22 to draw tissue 24 into ring 10. Alternatively, other techniques (such as pinching by hand or by a pinching tool) are used to draw the tissue into ring 10.

Once tissue 24 is firmly secured within ring 10, good acoustic coupling between the tissue and the ring is typically verified, prior to ultrasound device 8 entering a monitoring mode, for example, by transmitting “scout” waves from one side of the ring to the other. Following the drawing of the tissue into ring 10, ultrasound waves 27 are transmitted from device 8 toward a treatment focus zone 25

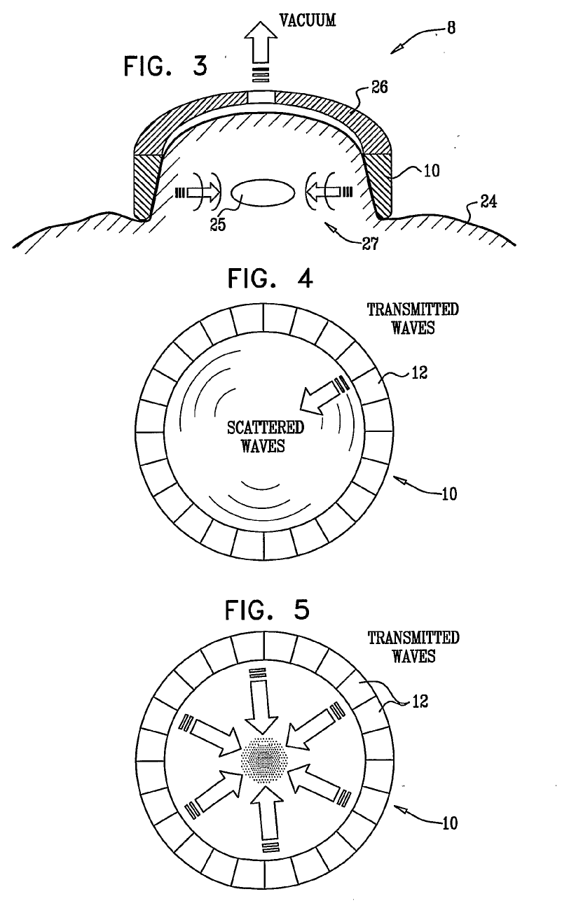

FIG. 4 is a schematic illustration of operation of ultrasound device 8 in a monitoring mode, in accordance with an embodiment of the present invention. One or more ultrasound transducers 12 transmit low energy waves into tissue 24 enclosed within ring 10. The resulting through-transmitted and/or scattered waves are typically detected by all of the ultrasound transducers (including the one or more transmitting ultrasound transducers), and are recorded for further analysis. The procedure is repeated using different transducers or signal parameters for transmission each time, until a sufficient amount of data is collected.

Maps of acoustic properties or images of the circular tissue area are reconstructed, typically using algorithms that are known in the art. As appropriate, the maps or images may depict various acoustic properties of the tissue, such as reflectivity, speed of sound, attenuation, acoustic impedance, and other properties. For some applications, the maps or images thus acquired are saved for later use as a reference set. In an embodiment, maps of acoustic properties are translated into maps that show tissue type within ring 10, and, for example, differentiate between fat tissue and muscle, nerve or blood cell tissues. Alternatively or additionally, maps of acoustic properties are translated into temperature maps, e.g., using techniques described in the above-cited PCT Publication WO 06/018837 to Azhari et al., which is incorporated herein by reference, and/or using other techniques known in the art. Further alternatively or additionally, maps of acoustic properties are assessed by computer or by a human to determine the efficacy of the treatment, and are saved or used to modify further treatments.

If an external source of energy is used to treat tissue 24 within ring 10, then ultrasound device 8 typically works only in the monitoring mode. Maps or images are typically acquired generally continuously during the treatment. The changes derived from the treatment result in changes of the detected acoustic properties of the treated tissue. By subtracting the new maps or images from the reference set of maps or images, the amount and location of damage is assessed. Alternatively, the reference set is not used, but instead a desired endpoint is designated, and a signal is generated when the endpoint is approached or attained.

In accordance with an embodiment of the present invention, ring 10 is switched to a treatment mode, typically a plurality of times in alternation with the monitoring mode described hereinabove. In the treatment mode, ultrasound transducers 12 transmit high intensity ultrasound waves, shock waves, sharp negative pressure pulses, continuous waves (CW), pulse sequences that cause cavitation, any other form of acoustical radiation that affects the tissue in a desired manner, or any combination of the above. Typically, but not necessarily, the ultrasound transducers transmit the energy in a HIFU mode.

FIG. 5 is a schematic illustration of the operation of ultrasound device 8 in a treatment mode, in accordance with an embodiment of the present invention. During, the treatment mode, some or all of ultrasound transducers 12 transmit high intensity waves simultaneously or in a temporal pattern, towards tissue 24 in the center of ring 10. This typically creates an imploding wave, such as an imploding cylindrical wave, whose amplitude (positive or negative) is high at the center. Consequently, damage to the tissue occurs relatively rapidly. Alternatively, other signal protocols create other ultrasound-based effects besides an imploding cylindrical wave, which, nevertheless, produce a desired level of tissue damage. In any case, following the transmission of the energy from transducers 12, ring 10 is typically switched back to the monitoring mode and damage assessment is performed. If appropriate, another iteration of high energy transmission is performed, followed by another iteration of monitoring. The procedure is repeated until satisfactory results are obtained. At this point, the vacuum is released under cover 26 and the operator or the robotic system moves the device to a new region to be treated, optionally based on feedback from the monitoring.

It is noted that by using phased array techniques, the phase of the transmitted waves from each ultrasound transducer 12 can be controlled such that the focal point of the imploding wave is moved over a significant portion of the area within ring 10, without physically moving the device. The timing of transmission of the ultrasound wave from each ultrasonic transducer 12 is set such that wave fronts transmitted from transducers 12 arrive to the focal point with generally the same phase, creating a sharp local peak in intensity which causes thermal and mechanical damage to tissue 24.

In some embodiments, a plurality of rings 10 are utilized in order to attain desired results.

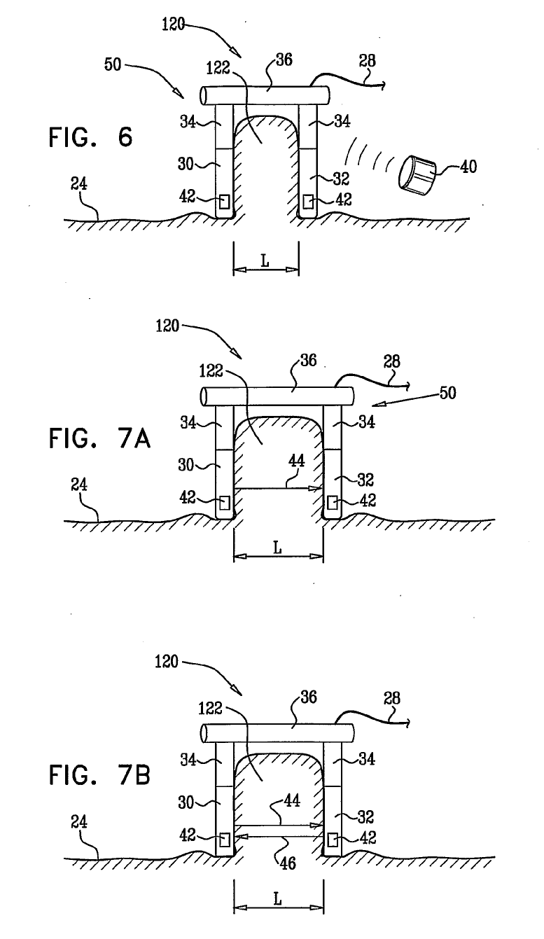

FIG. 6 is a schematic illustration of a system 120 for lipolysis and body contouring, comprising a housing 50, a plurality of acoustic elements comprising a subset 30 and a subset 32 of the acoustic elements, and an energy source 40, in accordance with an embodiment of the present invention. Each subset comprises one or more acoustic elements. At least a pair of acoustic elements are disposed at respective locations with respect to housing 50. Housing 50 is typically but not necessarily rigid, and comprises a support element 36 connected to ends of two cylinders 34 or members that are shaped in a different manner. In an embodiment, housing 50 is flexible, at least in part.

Subsets 30 and 32 are disposed upon cylinders 34, which are spaced at a distance L from one another. Distance L typically ranges from about 5 mm to about 150 mm, e.g., about 5 mm to 40 mm or 40 mm to 150 mm. The space between cylinders 34 defines a plane in which tissue 24 designated for treatment is drawn into housing 50. For some applications, an electromechanical system (not shown) is connected via lead 28 to support element 36 and moves cylinders 34 in a controlled motion, varying distance L between cylinders 34. When housing 50 is placed on tissue 24 designated for monitoring or treatment (as appropriate), such motion pinches and draws tissue 24 into the plane defined by housing 50. Alternatively or additionally, cylinders 34 rotate in the same direction or in opposite directions, to draw new tissue into the plane.

For some applications, the electromechanical system is disposed upon cylinders 34. For other applications, a source of suction, e.g., a vacuum pump disposed upon housing 50 draws a portion 122 of tissue 24 into housing 50.

Once tissue 24 has been drawn into housing 50, low intensity ultrasound energy used for detecting a parameter of portion 122 of tissue 24, e.g., fat content, is transmitted between first subset 30 and second subset 32. A first portion of first subset 30 transmits energy to be received, at least in part, by a first portion of second subset 32. Alternatively or additionally, a second portion of second subset 32 transmits energy to be received, at least in part, by a second portion of first subset 30. Cylinders 34 are arranged such that the energy is transmitted through portion 122 of tissue 24, e.g., typically parallel to the skin of the subject, and received on subset 30 and/or subset 32. Typically, tissue 24 includes skin of the subject and energy is transmitted from either subset 30 and 32, through the skin.

The electromechanical system maintains distance L between first subset 30 and second subset 32 during the monitoring and treatment process. Acoustic elements in subset 30 may be moved away from acoustic elements in subset 32 due to the movement of cylinders 34 by the electromechanical system. Alternatively or additionally, portions of the acoustic elements are moved to different locations with respect to cylinder 34. The movement and distances between the portions of the acoustic elements are typically recorded by a linear encoder or by counting steps of a stepper motor. Such recording is useful in the monitoring of the body contouring process, as described hereinbelow. Additionally, such recording is useful in facilitating calculating speed of sound (SOS), as described hereinbelow.

In an embodiment, the electromechanical system moves housing 50 to different locations on tissue 24 of the subject, enabling the acoustic elements to detect the presence of adipose tissue at multiple locations on tissue 24 of the subject. For some applications, moving housing 50 comprises rotating cylinders 34 along tissue 24 while periodically counter-rotating cylinders 34 such that tissue 24 is rolled between cylinders 34 and introduced within housing 50. Alternatively or additionally, the rolling of the cylinders is configured to induce a form of peristaltic motion of tissue 24. For other applications, the electromechanical system is not used to move housing 50 along tissue 24 of the subject.

Upon detection of the presence of adipose tissue, an independent energy source 40, that is not an acoustic element from subsets 30 and 32, applies treatment energy to portion 122 of tissue 24. Energy source 40 is coupled to housing 50 (configuration not shown), or, alternatively, mechanically separate from the housing. Energy source 40 comprises circuitry for focusing energy designated for the destruction of adipose tissue, such as acoustic energy (e.g., high intensity focused ultrasound, shock waves, sharp negative pressure pulses, or high intensity ultrasound waves), electromagnetic radiation (e.g., microwave radiation), laser energy, and/or visual or near-visual energy (e.g., infra-red). Energy source 40 transmits energy intense enough to cause damage to adipose tissue within portion 122. Effects or combined effects of treatments by energy source 40 may include, as appropriate, heating, tissue damage, thermal ablation, mechanical irritation, acoustic streaming, cell structure alteration, augmented diffusion, and/or a cavitation effect. For some applications, lipolysis is accomplished when energy source 40 elevates the temperature of portion 122 of tissue 24 by less than 10 C, e.g., less than 5 C.

For some applications, energy source 40 provides energy such that the treatment generates a combined effect of at least two of the above mentioned effects. For this application, energy is applied, inducing a different type of damage to the tissue. The sets are typically operated in a synchronized mode to enhance the tissue damaging process. Alternatively, a multipurpose array is used which is capable of producing at least two types of damage to a predefined tissue region by applying a plurality of transmissions (e.g., a sequence of transmissions or parallel transmissions). Inducing the at least two types of damage simultaneously or alternately creates synergism, accelerating the tissue damaging procedure and reducing the overall treatment time.

Energy source 40 transmits treatment energy in conjunction with the monitoring of the treatment process by acoustic subsets 30 and 32. For some applications, in addition to monitoring the treatment procedure, the body contouring process is tracked by sensors 42. For example, sensors 42 may comprise electromagnetic sensors or optical sensors that are coupled to housing 50. The sensed information is transmitted to a processing unit. Storing the tracking information allows for improved follow-up and comparison of body contouring treatments conducted on different days or during the treatment.

For some applications, tracking the treatment process occurs in conjunction therewith. In response to an indication of fat content detected by the acoustic elements in a particular area of the body of the subject, the a pre-treatment map is generated and the physician marks the area, designating it for treatment. Housing 50 is subsequently placed on the designated area to provide treatment and monitoring thereof. Following the treatment, housing 50 is re-positioned in the designated area to enable tracking of the body contouring process by sensors 42. Sensors 42 help ensure that (1) treatment has been applied to all subsections of the designated area and/or (2) treatment has not been applied multiple times to the same subsection during a single session. Thus, for some applications, treatment locations during one session are stored to facilitate the initiation of treatments in subsequent locations other than already-treated regions.

Through-transmitted and scattered waves are received by at least a portion of the acoustic elements. In some cases, the received waves are reflected from some acoustic elements towards other acoustic elements, which transfer information representing the detection of adipose tissue and subsequent monitoring of the treatment procedure to a processor (not shown). In an embodiment, the processor displays the information representing the detection by the acoustic elements, as well as provides on-line monitoring during the use of system 120.

Typically, monitoring by the acoustic elements is accomplished by a series of low intensity ultrasonic pulses transmitted from a portion of acoustic elements of subset 30. It is to be noted that other waveforms can be utilized. The energy is scattered by, reflected by, or transmitted through portion 122 of tissue 24. At least a portion of the energy is then received by subset 32, which is designated for monitoring the procedure. This portion of the energy is received by a portion of the acoustic elements of subset 32, and travel times of pulses between subset 30 and 32 (T₁) are calculated, using techniques known in the art. The amplitudes (Amp₁) of echoes received by the portion of the acoustic elements of subset 32 are also registered.

In like manner, for some applications, energy is transmitted from portions of the acoustic elements of subset 32 to be received and registered by portions of the acoustic elements of subset 30.

The average speed of sound (SOS) is calculated as follows:

SOS=L/T₁, where L represents the distance between subsets 30 and 32.

Distance L is recorded by a linear encoder, a stepper motor or another device known in the art and configured to sense and digitize linear position change for position measurement and feedback to the monitoring system, in order to calculate and monitor the SOS.

The average attenuation coefficient (mu) is calculated as follows:

mu=Log(Amp₁/Amp₀),

where Amp₀ is a reference amplitude.

In addition, the spectrum of both reflected waves (S_{R}) and the spectrum of the transmitted waves (S_{T}) are analyzed.

Using the properties SOS, mu, S_{R} and S_{T}, portion 122 of tissue 24 is characterized to assess whether the concentration of fat in portion 122 is sufficient for application of treatment energy thereto. Once treatment energy has been applied to portion 122, changes in the properties of SOS, mu, S_{R} and S_{T} are monitored. Expected changes as a result of the treatment process (e.g., elevated temperature, appearance of cavitation bubbles, and changes in the cellular structure) are manifested in and alter the acoustic properties SOS, mu, S_{R} and S_{T}. For example, it is known that SOS and mu change with temperature, and that the appearance of cavitation bubbles induces half harmonic signals in the spectrum relating to the reflected and transmitted waves. Methods described herein may be practiced in combination with methods for assessing a parameter of tissue described in the above-mentioned articles by Moran et al. and Akashi et al.

The portion of the acoustic elements of subset 32 which receive the scattered and through-transmitted echoes comprise transducers that are typically connected to a processing unit of a workstation (not shown). The workstation is configured to drive and receive data from the transducers. The workstation processes signals from the transducers in order to generate acoustic maps or images (e.g., a local B-scan image generated by the echoes or an image generated by through-transmission) of portion 122 of tissue 24 that is enclosed in the plane. The resulting maps or images indicate whether a desired extent of treatment has been obtained (e.g., a level of damage to tissue 24) and guide further treatment. Cycles of treatment and monitoring occur in a generally closed-loop manner and are repeated using different signaling parameters, until a sufficient amount of data is collected. Maps of acoustic properties or images of the tissue are reconstructed and assessed, as described hereinabove with reference to FIG. 4.

It is to be noted that since tissue 24 to be treated includes adipose tissue, for some applications the registered information is calibrated to provide tables relating the intensity of treatment to the expected changes in each of the acoustic properties: SOS, mu, S_{R} and S_{T}. When the desired effect has been achieved, the treatment is terminated.

Reference is now made to FIG. 7A, which is a schematic illustration of system 120, in accordance with another embodiment of the present invention. Acoustic elements detect the presence of fat and monitor the body contouring process as described hereinabove with reference to FIGS. 1 and 6. Subset 30 transmits the treatment energy to portion 122 of tissue 24 (as indicated by arrow 44). Subset 30 and/or 32 serves as a monitor and transmits signals for assessing the condition of portion 122 clamped between cylinders 34. Treatment by subset 30 is similar to the treatment effected by energy source 40, described hereinabove with reference to FIG. 6. Subset 30 and subset 32 work in conjunction with each other in a generally closed-loop operation cycling repeatedly between (a) subset 30 applying a treatment to portion 122 of tissue 24 in response to the monitored state of portion 122, and (b) subset 30 and/or 32 monitoring the state of portion 122 of tissue 24 following (a).

Reference is now made to FIG. 7B, which is a schematic illustration of system 20 of FIG. 7A with the exception that a first portion of acoustic elements of subset 32 transmits treatment energy (as indicated by arrow 46) in combination with the treatment energy transmitted by a first portion of the acoustic elements of subset 30 (as indicated by arrow 44), in accordance with an embodiment of the present invention. For some applications, portions of subsets 30 and 32 are activated simultaneously to generate a standing wave in the plane. The intensity peak of such a wave is located between subsets 30 and 32, and its frequency and amplitude are suitable for treating portion 122 of tissue 24. The same or other portions of subsets 30 and 32 monitor waves transmitted through or reflected from portion 122, typically between successive treatments by subsets 30 and 32.

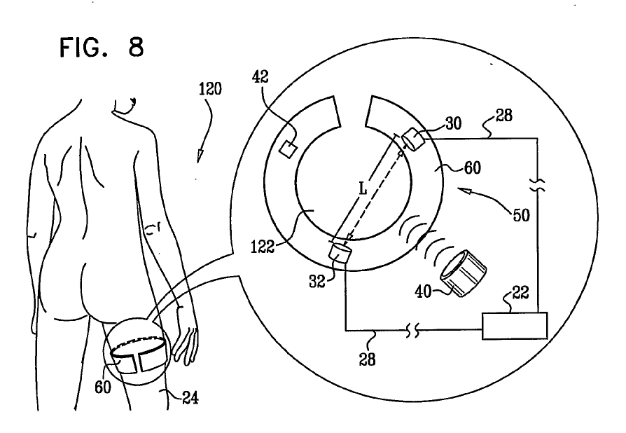

Reference is now made to FIG. 8, which is a schematic illustration of system 120 as described hereinabove with reference to FIG. 6, with the exception that housing 50 comprises a cuff 60, in accordance with an embodiment of the present invention. Cuff 60 is typically but not necessarily flexible and is designed to surround a limb of the subject, e.g., cuff 60 is shaped to provide a diameter of about 6 cm. For some applications, cuff 60 comprises a water bag that is designed to surround the limb to be treated. A plurality of acoustic elements are disposed with respect to cuff 60 so as to define a ring. In an embodiment, the “ring” comprises only two acoustic elements. Typically, the plurality of acoustic elements comprises between 2 and 64, e.g., 2 to 12, acoustic elements. Ultrasound waves are transmitted from a portion of acoustic elements of subset 30 through the limb and are received by a portion of acoustic elements of subset 32.

As shown, treatment energy is applied by independent energy source 40 as described hereinabove with reference to FIG. 6. As appropriate, energy source 40 may be mechanically independent of cuff 60, or mechanically coupled to the cuff (configuration not shown).

Typically, subset 30 is spaced apart from subset 32 at a distance L. Each subset 30 and 32 is typically but not necessarily connected to electromechanical system 22 via lead 28. Typically, electromechanical system 22 enables monitoring of different locations of the limb. Electromechanical system 22 varies distance L between subsets 30 and 32 by moving portions of the subsets to different locations on cuff 60. Distance L is recorded by a linear encoder or other device known in the art, in order to calculate and monitor the speed of sound as described hereinabove.

Alternatively or additionally, electromechanical system 22 moves both subsets clock-wise or counter-clockwise with respect to cuff 60, maintaining distance L constant. For some applications, subsets 30 and 32 are disposed at fixed locations upon cuff 60 and electromechanical system 22 rotates cuff 60 around the limb in order to position the subsets at different locations with respect to the limb.

For some applications, during the detecting of the concentration of fat in the limb, the acoustic elements of subsets 30 and 32 are moved such that two images of the limb are obtained. The first image is reconstructed from the reflected echoes depicting a standard B-scan image, and the other image is reconstructed from the through-transmitted waves, using ultrasonic tomography algorithms known in the art, e.g., Back-Projection-based methods. The second image may depict a map of SOS and/or mu in the imaged region.

For some applications, system 20 scans the limb and provides maps of tissue 24 before and after the treatment. In an embodiment, the treatment procedure is applied by a robotic system to the entire limb.

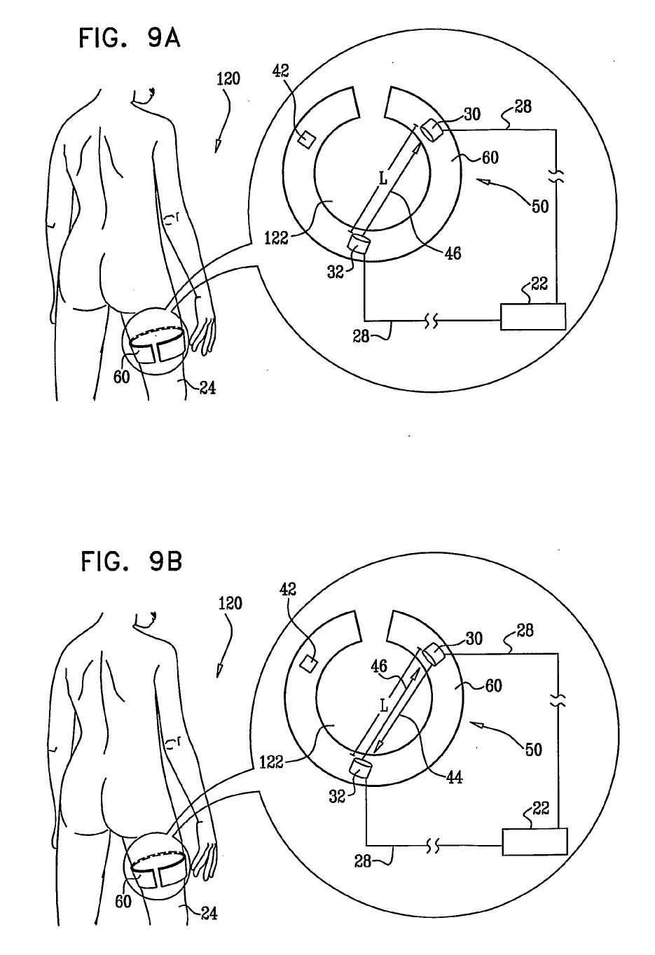

Reference is now made to FIGS. 9A and 9B, which are schematic illustrations of system 120 similar to the embodiments described hereinabove with reference to FIGS. 7A and 7B, respectively, with the exception that housing 50 comprises cuff 60. In FIG. 9A, treatment energy is transmitted only from subset 32 to subset 30 (as indicated by arrow 46), and in FIG. 9B, treatment energy is transmitted in both directions (as indicated by arrows 44 and 46).

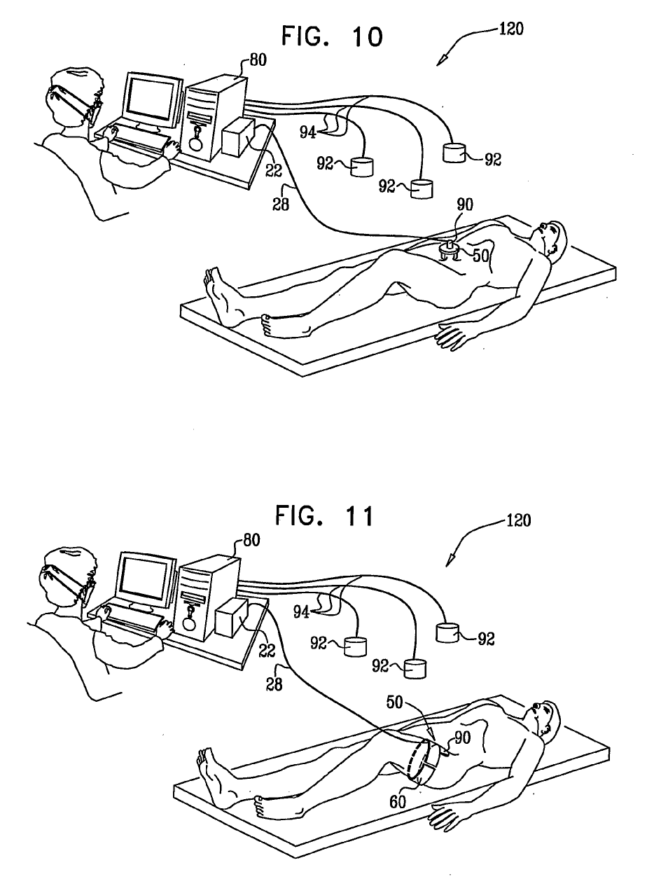

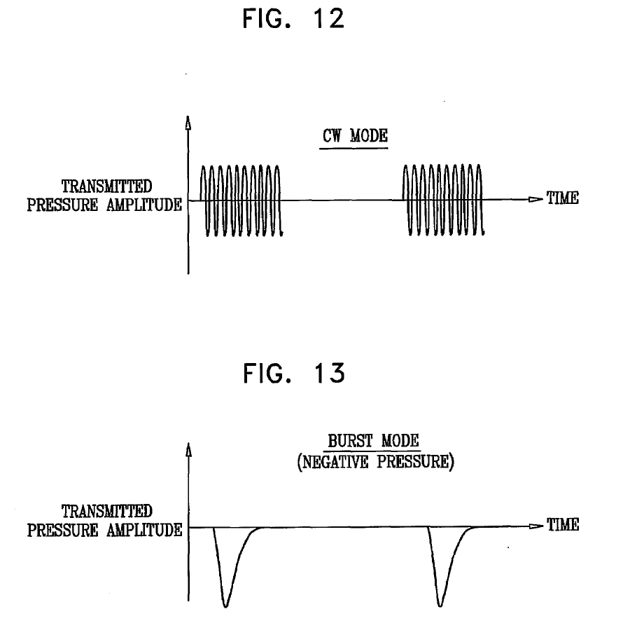

FIGS. 10 and 11 show system 120 comprising a tracking system comprising a plurality of reference sensors 92, in accordance with an embodiment of the present invention. Reference sensors 92 can be implemented in combination with each of the described embodiments of FIGS. 1-9B, and assess the location of treated tissue 24 by registering the relative spatial coordinates of the acoustic elements and/or anatomy of the patient. The sensed information is transmitted to a processing unit 80 by leads 94 coupled to reference sensors 92. Storing the location of treated areas allows for improved follow-up and comparison of treatments conducted on different days. For some applications, location sensing is performed in conjunction with the treatment to help ensure that (1) treatment has been applied to all subsections of a designated area, and/or (2) treatment has not been applied multiple times to the same subsection during a single session. Thus, for some applications, treatment locations during one session are stored, to facilitate treatments in subsequent locations being initiated outside of already-treated regions.

Typically, housing 50 comprises a sensor 90 in communication with reference sensors 92. For some applications, reference sensors 92 are placed at predetermined locations in the treatment room. Spatial localization of housing 50 with respect to coordinates of the room is achieved when reference sensors 92 transmit signals to sensor 90 (or vice versa, or when a spatial relationship is determined between sensors 92 and sensor 90). The localization can be based on measurements using electromagnetic waves (e.g., RF-induced currents in mutually-perpendicular coils), optical information (e.g., by processing video acquired by each of sensors 92) or acoustic waves (e.g., by time-of-flight measurements). In an embodiment, sensor 90 receives signals and transmits signals back to reference sensors 92 (or vice versa). The signals are subsequently transmitted to processing unit 80. For some applications, the signals transmitted from reference sensors 92 form an electromagnetic field around the patient, capable of being sensed by sensor 90. In such an embodiment, sensor 90 communicates either actively or passively with reference sensors 92, (e.g., passive communication may utilize radio frequency identification techniques known in the art).

For some applications, reference sensors 92 are placed at predetermined locations on the body of the subject (e.g., sternum, patella, pelvis, navel, etc.), and spatial localization of the housing and treated tissue relative to the anatomical landmarks is achieved.

In some embodiments, the spatial localization procedure is initiated by an operator, e.g., using a wand comprising reference sensor 92. The operator contacts predetermined anatomical landmarks of the patient and references the coordinates thereof with respect to housing 50.

For some applications, the spatial location of housing 50 during the treatment procedure is automatically registered along with other details such as intensity and duration of each treatment stage. This information is stored in processing unit 80 and used in following sessions as a reference for monitoring the treatment process.

For some applications, the physician may choose to save the obtained mapping information in the system memory of processing unit 80. In such a case, the spatial map may be recorded and graphically presented on a suitable display device. In an embodiment, graphical overlay of the spatial map generated during the treatment procedure is superimposed upon the pre-treatment map, thus indicating the damage to the tissue effected by the treatment procedure. For some applications, when the treatment required for the tissue region has been completed, an ink or other marking is stamped on the patient's skin, and the vacuum suction is then released. When the operator moves the device to a second region on the skin, its spatial orientation relative to the previously treated region, i.e., the marked region, is typically displayed via an electronic display. Alternatively or additionally, the spatial orientation of the second region is viewed in comparison with the marked area without the use of an electronic display, thus allowing the operator to monitor the progress of the entire session during the treatment procedure.

Reference is now made to FIGS. 12 and 13, which are graphs of transmitted pressure amplitude with respect to treatment time, in accordance with an embodiment of the present invention. As shown in FIG. 12, transmission is performed in a continuous wave (CW) mode, where a relatively long train of a sinusoidal wave is transmitted. FIG. 13 shows ultrasonic transmission in a burst mode, in which a sharp pulse is transmitted. The acoustic elements transmit ultrasound energy at a frequency of, typically but not necessarily, about 250 kHz.

Reference is again made to FIG. 12. In some embodiments, heating and cell implosion are effected as a result of the treatment procedure. For this particular application, the acoustic elements transmit ultrasound energy at a high frequency range of about 1-5 MHz, e.g., 3 MHz) in the CW mode. Such transmission heats portion 122 of tissue 24 to a relatively-high temperature of about 40-70 C, e.g., 45 C. In an embodiment, the temperature is evaluated using techniques described in PCT Publication WO 06/018837 to Azhari, which is incorporated herein by reference.

Reference is again made to FIG. 13. After reaching the desired temperature, high intensity implosion waves obtained using the burst mode are inwardly-directed to portion 122 of tissue 24. Although FIG. 13 depicts the pulse as having a negative amplitude, it is to be noted that bursts of pulses with positive amplitudes may be applied to tissue 24 of the subject. For some applications, the inwardly-directed wave creates a negative pressure pulsed wave. In such a case, a strong and rapid decrease in pressure at the focal point is created. As a result, tissue cells and/or connective tissue are subjected to tearing stresses causing irreversible damage thereto.

Once reference pre-treatment maps have been acquired, as described hereinabove with reference to FIG. 6, treatment is applied, typically using transmissions of short bursts (FIG. 13).

It is to be noted that embodiments of the present invention may be applied to treatments such as lipolysis and body contouring, face-localized molding of adipose tissue, mentoplasty and neck lift. For face, chin, and neck treatments, a small probe shaped to define a diameter of between about 2-4 cm is typically used. Other applications such as hair removal may be effected by using elements that create a linear focal zone in combination with some of the treatment procedures described hereinabove. For some applications, one element creates the linear focal zone. Alternatively, multiple elements are used to create several linear focal zones.

It is noted that although some embodiments of the present invention are described with respect to the use of ultrasound, the scope of the present invention includes replacing the ultrasound transducers described herein with transducers of other forms of energy, such as electromagnetic radiation.

Embodiments of the present invention described herein may be used, for example, for cosmetic purposes, such as by placing ultrasound device 8 and/or subsets 30 and 32 in contact with skin of the patient and treating tissue. The scope of the present invention includes application of the techniques described herein to tissue other than skin, as well. For example, ultrasound device 8 may be sized for placement during surgery on an intrabody organ of the subject, such as the heart or an abdominal organ.

It is to be noted that the scope of the present invention includes transmitting energy to slightly beneath the plane defined by the housing, e.g., up to 5 or up to 10 degrees beneath the plane defined by the housing, in order to treat tissue in the vicinity of the housing.

For some applications, techniques described herein are practiced in combination with techniques described in one or more of the references cited in the Cross-references section or Background section of the present patent application, which are incorporated herein by reference.

It is noted that although some embodiments of the present invention are described with respect to the use of ultrasound, the scope of the present invention includes replacing the ultrasound transducers described herein with transducers of other forms of energy, such as electromagnetic radiation.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-158. (canceled)

159. Apparatus, comprising:
a housing configured for placement on skin of a subject, and to draw at least a portion of the skin and underlying tissue within at least a part of the housing;
at least one ultrasound transducer coupled to the housing and configured to transmit through the skin and the underlying tissue one or more forms of acoustical radiation energy in accordance with one or more modes, at least one of the one or more forms of energy including treatment energy;
at least one acoustic element coupled to the housing and disposed with respect to the ultrasound transducer such that the one or more forms of energy is through-transmitted toward the acoustic element, at least a portion of the through-transmitted energy being received at least one component selected from the group consisting of: the at least one ultrasound transducer and the at least one acoustic element; and
a processing unit, configured to monitor a change in a parameter of the tissue underlying the skin, responsively to the received energy.

160. The apparatus according to claim 159, wherein the housing is configured to use suction to draw within the at least a part of the housing the portion of the skin and the underlying tissue.

161. The apparatus according to claim 159, wherein the ultrasound transducer is configured to transmit energy in a plane defined by the housing.

162. The apparatus according to claim 159, wherein the processing unit is configured to detect adipose tissue in the portion of the skin and the underlying tissue.

163. The apparatus according to claim 159, wherein the ultrasound transducer is configured to transmit the energy at an angle that is less than 10 degrees with respect to a lower surface of the apparatus.

164. The apparatus according to claim 159, wherein the processing unit is configured to generate a computed tomography (CT) image of the tissue in response to the energy received by the acoustic element.

165. The apparatus according to claim 159, wherein, in response to the through-transmitted energy received by the acoustic element, the processing unit is configured to monitor acoustic properties of the tissue and generate a temperature map based on the acoustic properties of the tissue.

166. The apparatus according to claim 159, wherein the ultrasound transducer comprises a first ultrasound transducer, and wherein the acoustic element comprises a second ultrasound transducer configured to receive at least a portion of the through-transmitted energy from the first ultrasound transducer.

167. The apparatus according to claim 159, wherein the apparatus is configured to configure the treatment energy to generate a standing wave in the tissue.

168. The apparatus according to claim 159, wherein the apparatus is configured to configure the treatment energy to generate an imploding wave in the tissue.

169. The apparatus according to claim 159, wherein:
the ultrasound transducer comprises a first ultrasound transducer,
the acoustic element comprises a second ultrasound transducer configured to transmit one or more forms of energy through the tissue, the one or more forms of energy including treatment energy,
the first ultrasound transducer is configured to receive at least a portion of the one or more forms of energy transmitted from the second ultrasound transducer, and
the processing unit is configured to monitor a change in the parameter of the tissue responsively to the energy received by the first ultrasound transducer from the second ultrasound transducer.

170. The apparatus according to claim 159, wherein:
the acoustic element comprises an ultrasound reflector configured to reflect the through-transmitted energy transmitted from the ultrasound transducer,
the ultrasound transducer is configured to receive at least a portion of the reflected energy, and
the processing unit is configured to monitor a change in the parameter of the tissue responsively to the reflected energy.

171. The apparatus according to claim 159, wherein:
the ultrasound transducer comprises a plurality of ultrasound transducers,
the plurality of ultrasound transducers is disposed in a given relationship with respect to the housing in which at least a first one of the plurality of transducers is disposed opposite at least a second one of the plurality of ultrasound transducers, and
a first portion of the plurality of ultrasound transducers is configured to transmit the energy toward at least one focus zone within a plane defined by the housing.

172. The apparatus according to claim 171, wherein the plurality of ultrasound transducers is disposed with respect to the housing so as to define a portion of at least one or more shapes selected from the group consisting of: a ring and an ellipse.

173. The apparatus according to claim 171, wherein the first portion of the plurality of ultrasound transducers is configured to transmit the energy in a manner in which the focal zone of the energy is moved over at least a portion of the plane defined by the housing.

174. The apparatus according to claim 171, wherein a second portion of the plurality of ultrasound transducers is configured to receive at least a portion of the energy transmitted from the first plurality of ultrasound transducers, and wherein the processing unit is configured to generate a computed tomography (CT) image of the tissue in response to the energy received by the second plurality of ultrasound transducers.

175. The apparatus according to claim 159, wherein:
the housing comprises at least first and second generally-parallel support structures configured to be disposed substantially perpendicularly with respect to a surface of skin surrounding the portion of the skin and the underlying tissue within at least a part of the housing,
the ultrasound transducer and the acoustic element are coupled to the first and second support structures, respectively, and
at least one of the support structures is movable with respect to the other support structure after the housing comes in contact with the tissue.

176. The apparatus according to claim 175, wherein the processing unit is configured to sense and digitize a position change of the at least one of the support structures in response to movement of the at least one of the support structures, and responsively thereto, to facilitate monitoring of the parameter of the tissue underlying the skin.

177. The apparatus according to claim 175, wherein the housing is configured to pinch the portion of the skin and the underlying tissue between the first and second support structures.

178. The apparatus according to claim 177, wherein the first and second support structures comprise first and second cylinders, respectively, and wherein at least one of the cylinders is rotatable with respect to the other cylinder in order to draw the portion of the skin and the underlying tissue within the at least a part of the housing.

179. The apparatus according to claim 159, wherein:
the housing comprises an electromechanical system and at least first and second generally-parallel support structures configured to be disposed substantially perpendicularly with respect to the skin,
the ultrasound transducer and the acoustic element are coupled to the first and second support structures, respectively, and
the electromechanical system is configured to vary a distance between the support structures after the housing comes in contact with the skin.

180. The apparatus according to claim 179, wherein the first and second support structures comprise first and second cylinders, respectively, and wherein the electromechanical system is configured to rotate the cylinders after the housing comes in contact with the skin.

181. The apparatus according to claim 180, wherein the housing is configured to pinch the portion of the skin and the underlying tissue between the first and second support structures.

182. The apparatus according to claim 159, wherein, in response to the through-transmitted energy received by the acoustic element, the apparatus is configured to generate a map of acoustic properties of the tissue.

183. The apparatus according to claim 182, wherein the apparatus is configured to differentiate between tissue types of the subject.

184. The apparatus according to claim 159, wherein the ultrasound transducer and the acoustic element are configured to operate in generally closed looped operation.

185. The apparatus according to claim 184, wherein the processing unit is configured to monitor the alteration of the parameter and regulate the transmission of the treatment energy in response to the monitoring.

186. The apparatus according to claim 184, wherein the ultrasound transducer and the processing unit are configured to cycle repeatedly between (a) applying a treatment to the tissue in response to the monitored state of the tissue, and (b) monitoring the state of the tissue following (a).

187. A method, comprising:
placing on skin of a subject a housing coupled to:
at least one ultrasound transducer, the ultrasound transducer being configured to transmit through the skin one or more forms of acoustical radiation energy in accordance with one or more modes, at least one of the one or more forms of energy including treatment energy, and
at least one acoustic element being disposed with respect to the ultrasound transducer such that the one or more forms of energy are through-transmitted toward the acoustic element;
drawing at least a portion of the skin and underlying tissue within at least a part of the housing;
transmitting from the ultrasound transducer one or more forms of energy through the skin and the underlying tissue;
receiving at least a portion of the through-transmitted energy at least one component selected from the group consisting of: the at least one ultrasound transducer and the at least one acoustic element; and
monitoring a change in a parameter of the tissue underlying the skin responsively to the received energy.

188. Apparatus, comprising:
a cuff housing configured to surround at least a portion of a limb of a subject;
at least one ultrasound transducer coupled to the housing and configured to transmit through at least a portion of the limb one or more forms of acoustical radiation energy in accordance with one or more modes, at least one of the one or more forms of energy including treatment energy;
at least one acoustic element coupled to the housing and disposed with respect to the ultrasound transducer such that the one or more forms of energy is through-transmitted toward the acoustic element, at least a portion of the through-transmitted energy being received at least one component selected from the group consisting of: the at least one ultrasound transducer and the at least one acoustic element; and
a processing unit, configured to monitor a change in a parameter of tissue of the limb, responsively to the received energy.

189. The apparatus according to claim 188, wherein:
the at least one ultrasound transducer comprises a plurality of ultrasound transducers,
the plurality of ultrasound transducers is disposed in a given relationship with respect to the cuff housing in which at least a first one of the plurality of transducers is disposed opposite at least a second one of the plurality of ultrasound transducers, and
a first portion of the plurality of ultrasound transducers is configured to transmit the energy toward at least one focus zone within a plane defined by the housing.

190. The apparatus according to claim 189, wherein the plurality of ultrasound transducers is disposed with respect to the housing so as to define a portion of at least one or more shapes selected from the group consisting of: a ring and an ellipse.
